(19) United States
(12) Patent Application Publication
(10) Pub. No.: US 2018/0044626 A1
(43) Pub. Date: Feb. 15, 2018

(54) COATED DRIED ACTIVE YEASTS AND FOOD PRODUCTS CONTAINING THE SAME

(71) Applicant: DANSTAR FERMENT AG, Zug (CH)

(72) Inventors: Richard DEGRE, Quebec (CA); Jerome BROUZES, Montgiscard (FR)

(21) Appl. No.: 15/790,389

(22) Filed: Oct. 23, 2017

Related U.S. Application Data
(63) Continuation of application No. 12/448,083, filed on Oct. 29, 2009, now abandoned.

(30) Foreign Application Priority Data
Dec. 8, 2006 (FR) 06/10735

(51) Int. Cl. C12N 1/04; A23K 40/30; A23K 10/18

(52) U.S. Cl. CPC C12N 1/04; A23K 10/18; A23K 40/30

(57) ABSTRACT

The invention relates to yeasts comprising a protection coating made of one or more compounds which are inert relative to said yeasts, characterized in that the yeasts are dried active yeasts. Application in the preparation of food products for animals.

### BACKGROUND OF THE INVENTION

### Field of the Invention

The subject of the invention is coated active dry yeast and food products containing same.

### Description of the Related Art

It is known that some yeast strains are used as additives (probiotics) in animal feed.

Food products for animals are most commonly sold in ₜhe form of granules; the latter are obtained by passing a mixture, initially formulated in the form of meals or powders, of the various ingredients (cereals, legumes, mineral salts, vitamins, etc.) through a die, in the presence of steam. This operation causes the temperature of the mixture to rise, due both to the injection of steam and to the mechanical forces associated with the forced passage through the die. Under these conditions, the yeast are subjected to a stress which leads to a loss of viability.

The term “loss of viability” is intended to mean a decrease in the number of cells capable of forming colonies (Colony Forming Units, CFU) compared with the number of CFU present in the mixture before granulation. This number is representative of the dose of “viable” probiotic additive in the food product. It is considered by users to be necessary to adhere to the recommended dose in order to ensure that the probiotic additive is effective.

It is therefore desirable to limit this loss of viability during the production of the granules as much as possible, or even to avoid it completely.

In order to remedy these drawbacks, it has been proposed to distribute the food product in the form of meal, without granulation. This solution is not, however, satisfactory since granulation has, in addition, advantages that are sought after by manufacturers and users (stockbreeders), namely better digestibility, easier distribution and consumption, better hygiene quality, etc.

Other solutions have consisted in proposing to incorporate the additive (yeast) into the cooled granules. Such an incorporation is, however, rarely feasible in practice, since food factories are not generally equipped for this type of operation. Furthermore, there is a risk of separation of the elements of the mixture due to the large difference in size between granules and yeast particles.

In another approach, it has been proposed to protect the yeast cells against the thermal and mechanical stresses by coating the particles. This solution is mentioned in particular in document FR 00 03409 in the name of Lallemand and document WO 92/12234.

The loss of viability still remains considerable, however, in certain cases (high-temperature or high-pressure granulation, for example).

### SUMMARY OF THE INVENTION

The inventors have sought to provide a solution to the abovementioned problems and have directed their studies to dry yeast. These yeast are most commonly sold in the form of “Instant Dry Yeast” (IDY). There also exist on the market yeast known as “Active Dry Yeast” (ADY), obtained by a different drying process. These two forms and processes are well known to those skilled in the art of yeast production. The appearance of the particles is different depending on the process: cylindrical (“vermicelli”) for IDY, spherical (“beads”) for ADY. In addition, the IDY are produced from yeast strains that react more rapidly than those used for the ADY (Food and Agricultural Industry 1/95, pages 9.13.4-1).

The ADY were developed in order to overcome the drawbacks of the compressed yeast available until then. Compressed yeast, which contain from 26% to 32% of solids, have the drawback of not maintaining their quality over time, in particular under certain climatic conditions. The ADY do not have this drawback due to the fact that they are dried and to their solids content, which is greater than 80%, and even 90%.

However, during the drying, which requires several hours, the ADY lose an enormous amount of their activity and require rehydration before use by dissolving in hot water.

New forms of yeast were therefore proposed, namely the IDY (U.S. Pat. No. 3,843,800). The production of IDY makes use of another production process and requires only a limited drying time (of the order of a few tens of minutes). They thus have an increased activity compared with the ADY and can be used as they are, without any rehydration, in particular for bread-making and wine-making.

It so happens (although this is not the effect initially sought) that the ADY are often more resistant to granulation than the IDY. However, this difference is relatively modest.

On the other hand, and surprisingly, the inventors have noted that coating the ADY leads to a synergic effect on the resistance thereof to granulation and makes it possible to significantly reduce the yeast viability losses.

The invention therefore relates to novel forms of coated active dry yeast.

It also relates to food products for animals which comprise such forms of yeast.

According to another aspect, the invention is also directed toward an easily implemented method for obtaining such forms of yeast and food products.

The yeast of the invention are yeast with a protective coating made of one or more compounds which are inert relative to said yeast and are characterized in that they are active dry yeast (ADY).

The coating is advantageously formed from compounds chosen from fatty acids, sugars, natural or synthetic polymers, and proteins.

An advantageous coating is thus produced from a mixture of stearic acid and palmitic acid.

The proportion of coating agent (or “coating”) relative to the yeast is preferably from 10% to 90% of the total mass of the final product.

Said yeast are more especially Saccharomyces.

They are more particularly Saccharomyces cerevisiae.

The yeast defined above are more particularly in the form of beads.

The invention is also directed toward food products for livestock or pets, characterized in that they are in the form of granules and contain yeast as defined above, according to the amounts necessary for obtaining the desired probiotic effect.

Thus, for food products intended for livestock, said yeast are, for example, present in a proportion of from 100 to 1000 g/tonne, preferably from 300 to 700 g/tonne, and in particular of the order of 500 g/tonne.

According to another aspect, the invention is directed toward a method for obtaining yeast as defined above, characterized in that it comprises coating active dry yeast (ADY) which are in the form of spherical particles, with one or more compounds which are inert relative to said yeast, so as to form a protective layer.

The yeast thus coated are advantageously used to prepare a food product for livestock or pets. The invention is therefore also directed toward a method for preparing food products for animals, characterized in that it comprises a step of granulating a mixture, in the form of meal, of a food product with a yeast as defined above.

Other characteristics and advantages of the invention will be given in the following exemplary embodiments.

### DETAILED DESCRIPTION OF THE INVENTION

### Example 1

A probiotic yeast strain (Saccharomyces cerevisiae, strain deposited with the CNCM [French National Collection of Microorganism Cultures] under No. 1-1077) was produced according to two different methods, resulting in two types of particles: instant dry yeast (IDY) and active dry yeast (ADY); these two methods are described, for example, in Food and Agricultural Industry 1/95, pages 9.13.4-1 to 9.13.4-3, or more particularly, for the IDY, in U.S. Pat. No. 3,843,800.

The two types of particles were then coated with a mixture of stearic acid and palmitic acid (50/50% by weight) according to the method described in FR 00 03409.

The particles obtained, and also the uncoated ADY and IDY particles, were incorporated, in a proportion of 500 grams per tonne, into a food product for cattle having the following composition:

| Starting material | % by weight |
|---|---|
| Wheat bran | 35 |
| Corn | 26 |
| Soybean cake | 21 |
| Beetroot molasses | 10 |
| Dehydrated alfalfa | 5 |
| Calcium carbonate | 1.95 |
| Premix of vitamins and minerals | 1 |
| Yeast | 0.05 |

The mixture obtained, in the form of meal, was introduced into a Sogem granulation press, type T 90 60 cv, equipped with a die which has a through-diameter of 4.5 mm and a thickness of 45 mm. The granule output temperature was 85° C.

4 batches of food product, each of one tonne, were produced:

one batch containing the uncoated IDY yeast

one batch containing the uncoated ADY yeast

one batch containing the coated IDY yeast

one batch containing the coated ADY yeast.

The contents of viable yeast (CFU) were analyzed in the meals and the granules of each batch.

The results are given in the following table:

| Type of | CFU/g in | CFU/g in | % loss of |
|---|---|---|---|
| yeast | meal | granules | viability |
| Uncoated IDY | 1.2 × 10⁷ | 2.0 × 10⁴ | 99.8 |
| Uncoated ADY | 1.1 × 10⁷ | 1.6 × 10⁵ | 98.6 |
| Coated IDY | 5.6 × 10⁶ | 4.5 × 10⁵ | 92 |
| Coated ADY | 4.9 × 10⁶ | 3.9 × 10⁶ | 20 |

Examination of this table shows that only the coated ADY yeast does not undergo a high loss of viability at the end of the granulation step. The loss of viability in fact represents only 20%, whereas it reaches 92% with the coated IDY yeast. The synergic effect of the coating on the resistance to granulation is also apparent from the results obtained.

### Example 2

A probiotic yeast strain (Saccharomyces cerevisiae, strain deposited with the CNCM under No. I-1079) was produced according to two different methods, resulting in two types of particles: instant dry yeast (IDY) and active dry yeast (ADY).

The two types of particles were then coated with a mixture of stearic acid and palmitic acid (50/50% by weight) as in Example 1 above.

The coated particles obtained, and also the uncoated ADY and IDY particles, were incorporated, in a proportion of 0.1%, into a food product for pigs having the following composition:

| Starting material | % by weight |
|---|---|
| Barley | 38 |
| Wheat | 25 |
| Soybean cake | 25 |
| Wheat bran | 6 |
| Fat | 3 |
| Calcium carbonate | 1.9 |
| Premix of vitamins and minerals | 1 |
| Yeast | 0.1 |

The mixture obtained, in the form of meal, was introduced into a Kahl laboratory granulation press (type 14-175), equipped with a die which has a through-diameter of 4 mm. The granule output temperature was 82° C.

4 batches of food product, each of 50 kg, were produced:

one batch containing the uncoated IDY yeast

one batch containing the uncoated ADY yeast

one batch containing the coated IDY yeast

one batch containing the coated ADY yeast.

The contents of viable yeast (CFU) were analyzed in the meals and the granules of each batch.

The results are given in the following table:

| Type of | CFU/g in | CFU/g in | % loss of |
|---|---|---|---|
| yeast | meal | granules | viability |
| Uncoated IDY | 1.84 × 10⁷ | 7.9 × 10⁵ | 95.7 |
| Uncoated ADY | 2.02 × 10⁷ | 1.95 × 10⁶ | 90.3 |
| Coated IDY | 1.1 × 10⁷ | 2.15 × 1⁶ | 80.5 |
| Coated ADY | 1.13 × 10⁷ | 9.34 × 10⁶ | 17.3 |

As in Example 1, the resistance to granulation of the ADY yeast is greatly increased after coating and the percentage loss of viability is low compared with that observed with the coated IDY yeast.

By virtue of the specific choice of the ADY yeast, the invention provides means of great value for obtaining food products having notable probiotic properties.

1. A yeast with a protective coating made of one or more compounds which are inert relative to said yeast, wherein the yeast is an active dry yeast, commonly referred to as ADY.

2. The yeast as claimed in claim 1, wherein the coating is formed from compounds selected from the group consisting of fatty acids, sugars, natural or synthetic polymers and proteins.

3. The yeast as claimed in claim 1, wherein the coating is produced from a mixture of stearic acid and palmitic acid.

4. The yeast as claimed in claim 1, wherein a proportion of coating agent relative to the yeast is from 10% to 90% by weight of a total mass of a final product.

5. The yeast as claimed in claim 1, wherein the yeast is Saccharomyces.

6. The yeast as claimed in claim 1, wherein the yeast is Saccharomyces cerevisiae.

7. The yeast as claimed in claim 1, wherein the yeast is in a form of beads.

8. A food product for livestock or pets, wherein the food product is in a form of granules and contains, as probiotic additives, the yeast according to claim 1.

9. The food product as claimed in claim 8, wherein the food is adapted for livestock, and said yeast is present in a proportion of from 100 to 1000 g/tonne.

10. The food product as claimed in claim 8, wherein the food is adapted for livestock, and said yeast is present in a proportion of from 300 to 700 g/tonne.

11. The food product as claimed in claim 8, wherein the food is adapted for livestock, and said yeast is present in a proportion of an order of 500 g/tonne.

12. A method of obtaining the yeast as claimed in claim 1, comprising:
coating the active dry yeast which are in a form of spherical particles, with the one or more compounds which are inert relative to said yeast, so as to form a protective layer.

13. A method for preparing a food product for livestock or pets, comprising:
Granulating a mixture, in a form of meal, of a food product with a yeast as claimed in claim 1.
